(19) United States
(12) Patent Application Publication
(10) Pub. No.: US 2025/0256029 A1
(43) Pub. Date: Aug. 14, 2025

(54) LIGHT-BASED VISUAL CUEING OF MEDICATION DELIVERY INSTRUCTIONS USING LIGHT AND MOTION SENSORS

(71) Applicant: Janssen Research & Development, LLC, Raritan, NJ (US)

(72) Inventor: Jason TANSKY, Raritan, NJ (US)

(21) Appl. No.: 18/706,576

(22) PCT Filed: Oct. 17, 2022
(86) PCT No.: PCT/IB2022/059948

§ 371 Date: May 1, 2024

Related U.S. Application Data
(60) Provisional application No. 63/275,825, filed on Nov. 4, 2021.

(51) Int. Cl. A61M 5/20

(52) U.S. Cl. CPC A61M 5/20; A61M 2005/2006; A61M 2205/3306; A61M 2205/583

(57) ABSTRACT

Light-based visual cues may be provided, for example to assist a patient in proper use of a medication delivery device, such as a medication injection device or a medication storage container. A light source, such as an organic light-emitting diode (OLED), may be used, which, when activated, may be a visual cue to perform a medication delivery instruction and/or may illuminate medication delivery information. Some example instructions may be to remove a cap of a medication delivery device or to shake the medication delivery device. A light sensor, motion sensor, microphone and/or other sensors may also be employed to trigger activation of the light source, based on detected light, motion and/or sound.

### TECHNICAL FIELD

The present disclosure relates generally to providing light-based visual cues, for example to assist a patient in proper use of a medication delivery device. Light sources, such as one or more organic light-emitting diodes (OLED's), may be activated, for example based on output from one or more sensors.

### BACKGROUND

A medication delivery device may be used to store and/or deliver a medication. In some examples, a medication delivery device may be a medication injection device, such as a prefilled syringe, an autoinjector, or wearable infusion pump. In some other examples, a medication delivery device may be a medication storage container, such as a bottle, a blister pack or other medication packaging.

Patients may often not perform an injection properly, such as by failing to remove a cap of the medication delivery device, not sufficiently pressing down on a medication delivery device to inject a complete dosage, failing to lift the medication delivery device up after injection, and the like. Additionally, patients may sometimes neglect to perform other medication delivery steps, such as shaking a medication before use.

Medication delivery instructions may sometimes be printed on a product label or instruction sheet. However, users may often neglect to read the printed instructions or may not completely comprehend the instructions. Moreover, it may be unclear from printed instructions exactly which step of a process the user is currently on, thereby potentially resulting in missed, or otherwise improperly performed, steps and instructions.

### SUMMARY

An example light-based medication delivery cueing system is described. The example system may include a light sensor and a motion sensor. The light sensor may detect light corresponding to a removal of a medication delivery device from a storage component. The motion sensor may detect motion of the medication delivery device. The example system may further include a first light source that is activated based at least in part on a first detection of the light and a second detection of the motion. Activating of the first light source may be a first visual cue to perform a first medication delivery instruction. In some examples, the light sensor, the motion sensor and the first light source may be included in one or more adhesive labels that are attached to the medication delivery device. In some other examples, the light sensor, the motion sensor and the first light source may be included within the medication delivery device.

An example light-based medication delivery cueing method is described. The example method may include detecting, by a light sensor, light corresponding to a removal of a medication delivery device from a storage component. The example method may further include detecting, by a motion sensor, motion of the medication delivery device. The example method may further include activating, based at least in part on the detecting of the light and the detecting of the motion, a first light source. Activating of the first light source may be a first visual cue to perform a first medication delivery instruction. In some examples, the light sensor, the motion sensor and the first light source may be included in one or more adhesive labels that are attached to the medication delivery device. In some other examples, the light sensor, the motion sensor and the first light source may be included within the medication delivery device.

Another example light-based medication delivery cueing system is described. The example system may include a timer configured to detect an occurrence of a designated date. The example system may further include a light sensor that may detect light corresponding to a removal of a medication delivery device from a storage component. The light sensor may be powered based on a first detection of the occurrence of the designated date. The example system may further include a light source that is activated based at least in part on a second detection of the light. In some examples, the timer, the light sensor, and the light source may be included in one or more adhesive labels that are attached to the medication delivery device. In some other examples, the timer, the light sensor, the motion sensor and the light source may be included within the medication delivery device.

Another example light-based medication delivery cueing system is described. The example system may include a motion sensor that detects motion of a medication delivery device. The example system may further include a timer configured to detect an expiration of a designated time period. The example system may further include a first light source that is activated based at least in part on detection of the expiration of the designated time period. The example system may further include a second light source that is activated based at least in part on detection of the motion. In some examples, the timer, the motion sensor, the first light source and the second light source may be included in one or more adhesive labels that are attached to the medication delivery device. In some other examples the timer, the motion sensor, the first light source and the second light source may be included within the medication delivery device.

Another example light-based medication delivery cueing system is described. The example system may include a microphone that detects at least one detected sound. The example system may further include one or more processing components that determine first audio characteristics of the at least one detected sound and that detect a sound match between the first audio characteristics and second audio characteristics of least one injection completion sound that is made by the medication delivery device in association with a completion of an injection. The example system may further include a light source that is activated based at least in part on a detection of the sound match. In some examples, the microphone, the one or more processing components and the light source may be included in one or more adhesive labels that are attached to the medication delivery device. In some other examples the microphone, the one or more processing components and the light source may be included within the medication delivery device.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1A is a front elevation view of a first example medication delivery device in a pre-use position.

FIG. 1B is a front elevation view of the first example medication delivery device with the cap removed so as expose the needle guard.

FIG. 1C is a front elevation view of the first example medication delivery device with the needle guard moved from its position in FIG. 1B.

FIG. 1D is a front elevation view of the first example medication delivery device with the upper housing moving toward the dispensed position.

FIG. 1E is a front elevation view of the first example medication delivery device with the upper housing in the dispensed position.

FIG. 1F is a front elevation view of the first example medication delivery device with the needle guard in the final position.

FIG. 2 is a view of a second example medication delivery device that is an example pre-filled syringe.

FIG. 3 is a view of a third example medication delivery device that is an example autoinjector.

FIG. 4 is a view of a fourth example medication delivery device that is an example wearable medication delivery device.

FIG. 5 is a view of an example storage component that may store a medication delivery device.

FIG. 6 is a view of an example removal of a medication delivery device from an example storage component.

FIG. 7 is a view of a first example illuminable medication delivery instruction panel on the first example medication delivery device.

FIG. 8 is a view of the first example illuminable medication delivery instruction being illuminated in response to motion of the first example medication delivery device.

FIG. 9 is a diagram of an example light source that illuminates the first example medication delivery instruction panel.

FIG. 10 is a diagram of a first example light-based medication delivery cueing system.

FIG. 11 is a view of a second example illuminable medication delivery instruction panel on a fourth example medication delivery device.

FIG. 12 is a diagram of a second example light-based medication delivery cueing system.

FIG. 13 is a diagram of an example light-based medication delivery cueing process.

FIG. 14 is a diagram of a third example light-based medication delivery cueing system.

FIG. 15 is a diagram of a fourth example light-based medication delivery cueing system.

FIG. 16 is a diagram of a fifth example light-based medication delivery cueing system.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims. Further, the term “at least one” stated structure as used herein can refer to either or both of a single one of the stated structure and a plurality of the stated structure. Additionally, reference herein to a singular “a,” “an,” or “the” applies with equal force and effect to a plurality unless otherwise indicated. Similarly, reference to a plurality herein applies with equal force and effect to the singular “a,” “an,” or “the.”

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

### Example Medication Delivery Devices

Some example medication delivery devices will now be described in association with which the light-based visual cueing techniques described may be implemented. The term medication delivery device, as used herein, refers to a device that is used to store and/or deliver a medication. Medication delivery devices may include, for example, medication injection devices and medication storage containers. In some examples, the light-based visual cueing techniques described herein may be implemented in association with medication injection devices such as autoinjectors, prefilled syringes, wearable medication delivery devices (e.g., patch pumps, on body delivery systems (OBDS's), etc.), and others. It is noted, however, that the techniques described herein are not limited to use with any particular types of medication delivery devices, and a wide variety of types of medication delivery devices may be employed.

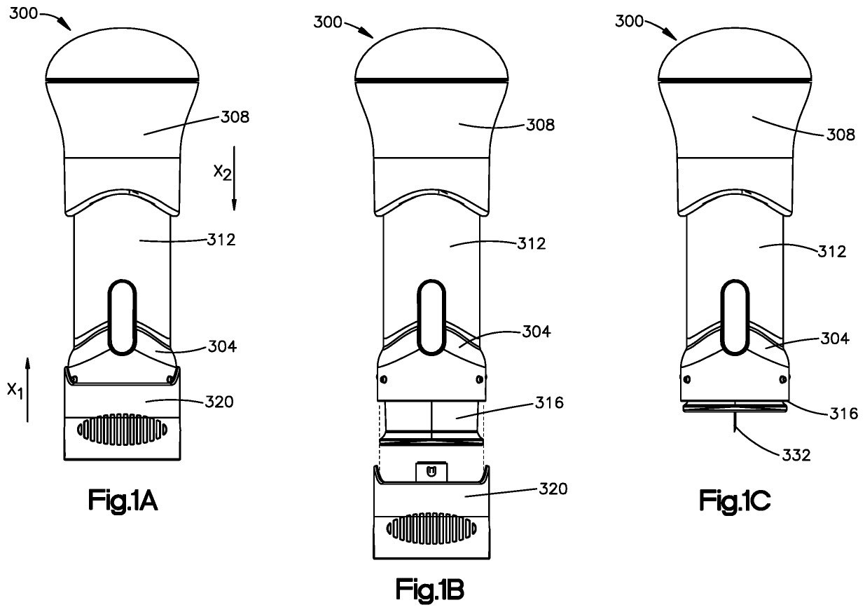

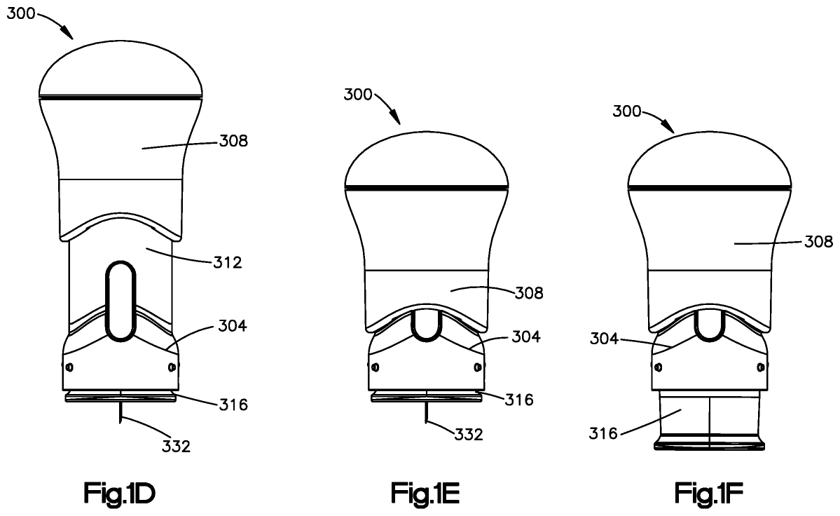

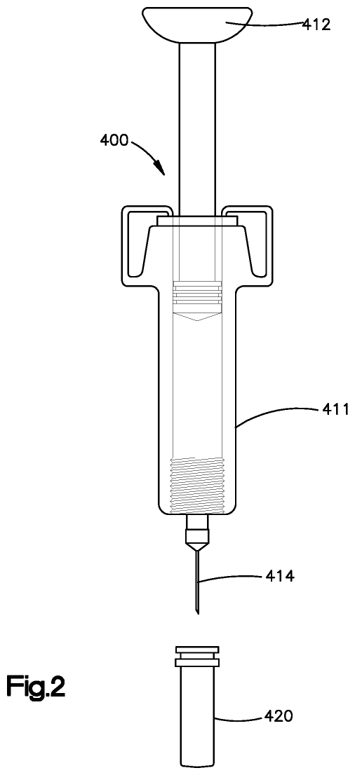

Referring now to FIGS. 1A-1F, a first example medication delivery device is described in association with which the light-based visual cueing techniques described may be implemented. As will be described, the device 300 may be employed to inject a medication, such as by pressing down on an upper housing 308 and moving the upper housing from a pre-use position to a dispensed position. In operation and in reference to FIGS. 1A-1F, the device 300 can be configured to deliver a medication. Prior to use, the upper housing 308 can be locked in the pre-use position, and the cap 320 can be coupled to the lower housing 304 so as to shield the needle guard 316 and the needle 332. When the device 300 is ready to be used, the cap 320 can be removed from the lower housing 304 as shown in FIG. 1B.

As shown in FIG. 1C, the device 300 can be positioned against a skin surface and a manual force can be applied to the upper housing 308 along an insertion direction, which is direction X2 in FIG. 1A, such that, as the needle guard 316 is pressed against the skin surface, the needle guard 316 moves and the needle 332 is inserted into the tissue. As the needle guard 316 moves, the upper housing 308 may be unlocked from the pre-use position. As shown in FIGS. 1D and 1E, the upper housing 308 can then be moved along the insertion direction and over the middle housing 312. The upper housing 308 of device 300 is supported relative to the lower housing 304 and is configured to receive a manual force and move with respect to the lower housing 304 in the insertion direction from the pre-use position to the dispensed position in response to the manual force. The device 300 may include an internal syringe that is supported by the lower housing 304 and a plunger rod that is carried by the upper housing 308 and movable with the upper housing 308 so as to advance relative to the syringe when the upper housing 308 is moved along the insertion direction. The syringe may retain a medication and carry the needle 332 that is configured to be inserted into tissue. Advancement of the plunger rod relative to the syringe may cause the syringe to deliver the medication out the needle 332 and into the tissue. When the upper housing 308 reaches the dispensed position, the upper housing 308 may be locked in the dispensed position, for example via internal locking latches and latch members, so as to prevent re-use of the device 300. As the upper housing 308 is locked in dispensed position, such as by locking latches snapping over the latch members, an audible click may be produced that signifies to the user that the upper housing 308 has reached the dispensed position and is locked in the dispensed position. The upper housing 308 can be permanently locked in the dispensed position such that the device 300 is not reusable. It should be appreciated, however, that the upper housing 308 can be temporarily locked such that the device 300 can be sterilized and reused.

As shown in FIG. 1F, when the device 300 is removed from the skin surface along a direction X1 opposite the insertion direction the needle guard 316 moves along the insertion direction to the final position. When in the final position, the needle guard 316 can be permanently locked in the final position so that the device 300 is not reusable. It should be appreciated, however, that the needle guard 316 can be temporarily locked such that the device 300 can be sterilized and reused.

Thus, device 300, which is described above with reference to FIGS. 1A-1F, is one example of a medication delivery device in association with which the light-based visual cueing techniques described herein may be implemented. However, the light-based visual cueing techniques described herein may also be implemented in association with other medication delivery devices, such as autoinjectors, prefilled syringes, wearable medication delivery devices (e.g., patch pumps, OBDS's), and others.

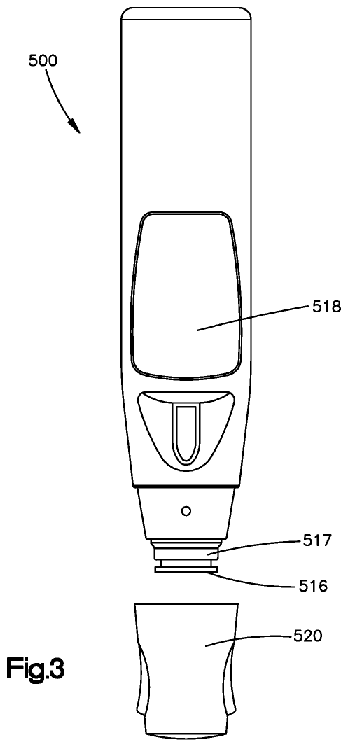

Referring now to FIG. 2, device 400 is an example of a prefilled syringe in association with which the light-based visual cueing techniques described herein may be implemented. As shown in FIG. 2, device 400 has a cap 420, which may be removed by the user when the user is ready to inject a medication using the device 400. After removing the cap 420, the device 400 may be held, and the needle 414 may be inserted. The user may then place his or her thumb on the plunger 412 and press the plunger 412 all the way down until the plunger 412 stops. The user may then release pressure from the plunger 412. The needle 414 may then retract into the body 411. Thus, in device 400, the user presses down on the plunger 412, for example as opposed to upper housing 308 of device 300 of FIG. 3.

Referring now to FIG. 3, device 500 is an example of an autoinjector in association with which the light-based visual cueing techniques described herein may be implemented. As shown in FIG. 2, device 500 has a cap 520, which may be removed by the user when the user is ready to inject a medication using the device 500. After removing the cap 520, the user may hold device 500 and position the device 500 against the skin with the safety sleeve 516 flat against the skin. The user may then push firmly against the skin, which may cause the safety sleeve 516 to slide into the cover 517. The user may then press button 518, which may cause a medication to be injected into the user via a hidden needle, such as may be located adjacent to safety sleeve 516 and cover 517. The device 500 may produce an audible sound, such as a first click sound, as the button 518 is pressed and the injection of the medication is started. The user may continue to press button 518 until the medication is fully dispensed. Similar to device 300 of FIG. 3, device 500 may also produce an audible sound, such as a click sound, once the medication has been fully dispensed, and this second click sound may be an indication to the user that the medication has been fully dispensed. Upon hearing the second click sound, the user may lift the device 500 from the skin. Thus, one way in which device 500 differs from device 300 is that device 500 allows the injection to be triggered by pressing a button 518.

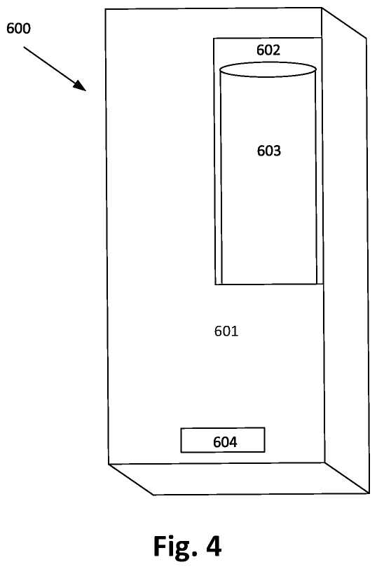

Referring now to FIG. 4, device 600 is an example of a wearable medication delivery device in association with which the light-based visual cueing techniques described herein may be implemented. Device 600 may be, or may be included in, an on body delivery system (OBDS). FIG. 4 shows a front surface 601 of device 600. In some examples, device 600 may be attachable to a user's skin. For example, in some cases, a rear surface of device 600, which may be opposite front surface 601 (and which is not shown in FIG. 4), may have an attached adhesive pad that may allow attachment of device 600 to a user's skin. Also, in some examples, device 600 may have an associated strap for assistance in attaching device 600 to a user's skin. As shown in FIG. 4, a medication tube 603 is visible through window 602. In operation, device 600 may cause a medication to be delivered from medication tube 603 to a user via an injection. In the example of FIG. 4, a user may press button 604 to cause the injection process to start. Device 600 may include a needle, such as may protrude from the rear of device 600, through which injection of the medication may be delivered.

Thus, as described above, the light-based visual cueing techniques described herein may be implemented in association with medication delivery devices such as autoinjectors, prefilled syringes, wearable medication delivery devices (e.g., patch pumps, on body delivery systems (OBDS's), etc.), and others. However, it is again noted that the techniques described herein are not limited to use with any particular types of medication delivery devices, and a wide variety of types of medication delivery devices may be employed. As another example, the light-based visual cueing techniques described herein may also be used in combination with other medication delivery devices, such as medication storage containers (e.g., storage bottles, blister packs and other storage packages, etc.).

### Light-Based Visual Cueing of Medication Delivery Instructions

In some examples, one or more light sources may be activated in order to cue a user to perform one or more medication delivery instructions. A medication delivery instruction is an instruction related to medication delivery, such as a remove cap instruction, a shake well instruction, a place on skin instruction, a press down instruction, a lift straight up instruction, or a check medication expiration date instruction. Activating a light source means that the light source is caused to assume one or more active states, such as to start emitting light (e.g., by being powered-on), to start flashing, to change color (e.g., to change to a green color or another designated color associated with an active state), and the like. In some examples, a light source may illuminate information corresponding to an instruction. The illuminated information may be, or may include, text, for example including words such as “remove cap” or “shake well”. In other examples, the illuminated information may be, or may include, a non-textual instruction, for example including one or more symbols, such as an arrow. For example, a vertical double-sided arrow could be displayed to instruct a patient to shake a medication delivery device. As another example, a horizontal double-sided arrow could be displayed to instruct a patient to remove a cap. In yet other examples, illuminated information may include a combination of text and symbols. For example, for a shake well instruction, the illuminated information could include a vertical double-sided arrow in combination with the words “shake well.” As another example, for a remove cap instruction, the illuminated information could include a horizontal double-sided arrow in combination with the words “remove cap.”

As described herein, illuminable information may be illuminated by a light source, such as may include an organic light-emitting diode (OLED), an OLED display, a light-emitting diode (LED), an LED display, other thin film type lights, incandescent lights, and/or other light sources. As also described herein, the light source may be activated (e.g., caused to emit light, caused to flash, and/or change colors) based on one or more sensors, such as a light sensor and/or a motion sensor. In one specific example, activation of a light source may be a visual cue to perform a medication delivery instruction, such as an instruction to remove a cap or instruction to shake a medication delivery device. The light source may be activated based on a combination of a light sensor, a motion sensor, and/or other sensors. The light sensor may detect light corresponding to a removal of the medication delivery device from a storage component. The motion sensor may detect motion corresponding to a user's movement of the medication delivery device. The light source may be activated based on detection of the light and/or detection of the motion. This and other example embodiments are described in detail below.

Patients and other users may often neglect to perform steps in the medication delivery process and/or perform steps improperly. For example, users may forget to check an expiration date, may forget to shake a medication container prior to use, may forget to remove a cap, etc. In other examples, users may perform these and other steps improperly. For example, users may not sufficiently shake a medication container for a required time period or may not perform an injection properly (e.g., may not press all the way down). By illuminating information corresponding to medication delivery instructions, the information may be more visible and may appear more important to users, thereby reducing the likelihood that users may forget to perform an instructed act and/or may not perform the instructed act properly.

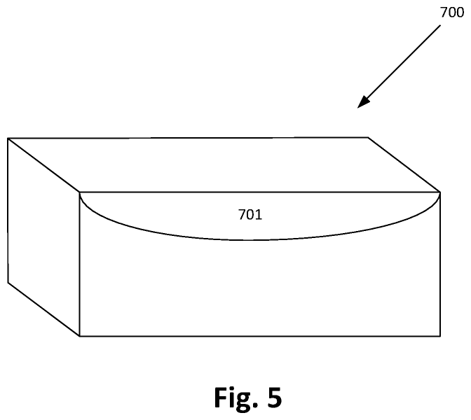

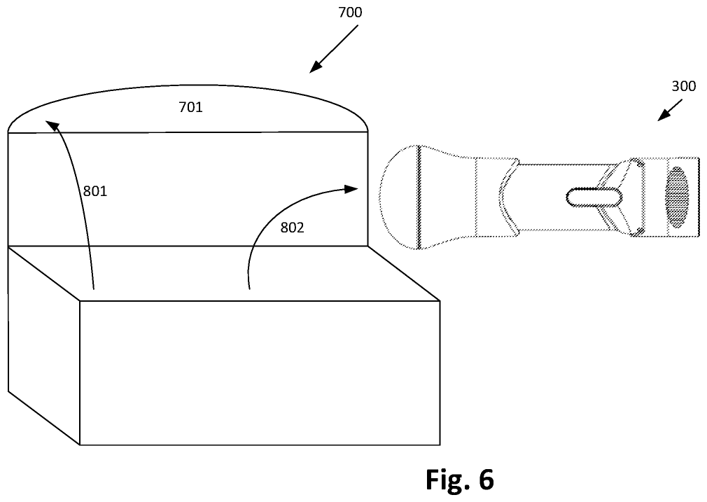

FIG. 5 shows an example storage carton 700, which stores device 300. Thus, storage carton 700 is one example of a storage component for storing a medication delivery device. Other examples of storage components may include containers, packages, wrappers, cases, and the like. In FIG. 5, storage carton 700 is closed with device 300 enclosed within the storage carton 700 (and therefore not visible in FIG. 5). In this example, the storage carton 700 may be opened, such as by lifting flap 701, for example as shown in FIG. 6. Referring now to FIG. 6, flap 701 is lifted by user such as in the direction depicted by arrow 801. The user may then remove the device 300 from the storage carton 700, such as by lifting the device 300 out from storage carton 700 in the direction depicted by arrow 802.

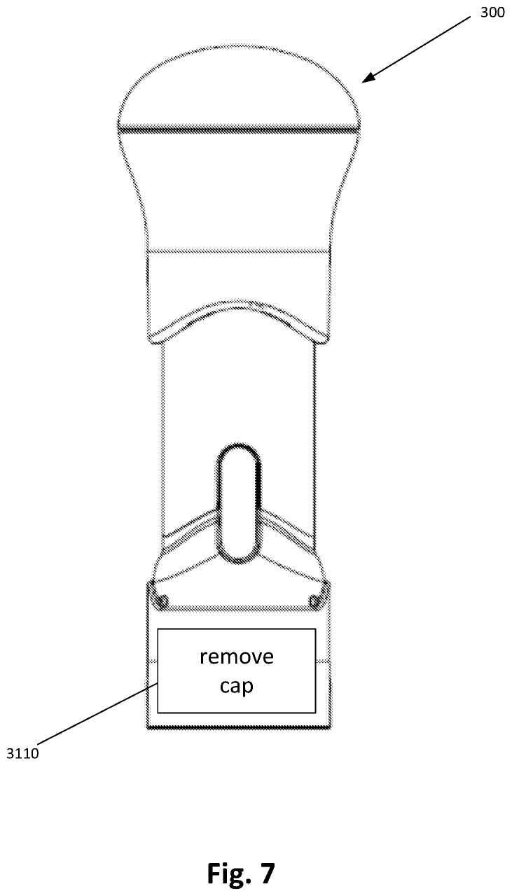

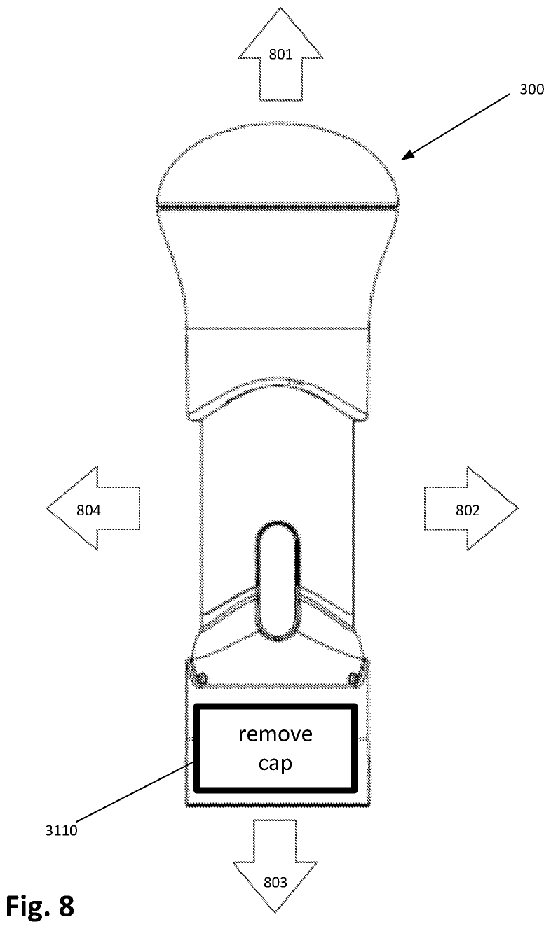

Referring now to FIG. 7, an example is shown of an illuminable instruction panel 3110 on device 300. In this example, the illuminable instruction panel 3110 displays the instruction “remove cap”. As will be described in detail below, the illuminable instruction panel 3110 may be illuminated based on detection of light, such as when the device 300 is removed from a storage component (e.g., storage carton 700 of FIGS. 5-6), and detection of motion, such as when the device 300 is moved by a user. FIG. 8 shows an example in which the device 300 is moved by a user, such as in one or more directions represented by arrows 801-804 and/or other directions. In the example of FIG. 8, the illuminable instruction panel 3110 has been illuminated in response to detected light (e.g., when the device 300 is removed from storage carton 700) and detected motion corresponding to the movement of device 300. The illumination of illuminable instruction panel 3110 in FIG. 8 is indicated by the thick, bold outlining of the illuminable instruction panel 3110 in FIG. 8 (as compared to the thinner outlining of the illuminable instruction panel 3110 in FIG. 7).

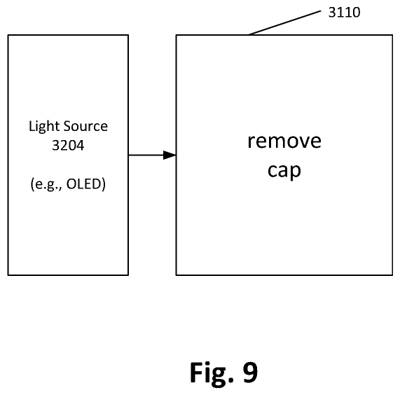

Referring now to FIG. 9, it is shown that the illuminable instruction panel 3110 may be illuminated by a light source 3204, which may also be included in device 300. For example, in some cases, the light source 3204 may be positioned directly underneath the illuminable instruction panel 3110. In order to not cause substantial changes to the size and/or shape of the device 300 described above, the light source 3204 (as well as other light sources described herein) may be a thin, flat and/or flexible component, such as an OLED and/or OLED display. In some examples, for the light sources described herein, other light sources may be employed, such as an LED, an LED display, other thin film type lights, incandescent lights, and/or other light sources. In some examples, to result in enhanced visibility to the patient, the light source 3204 may have a color that differs from the color of the exterior of the device 300. For example, in some cases, the exterior of device 300 could have a white color, and the light source 3204 could have a red, yellow or green color.

In some examples, the illuminable instruction panel 3110 may be substantially translucent. By being substantially translucent, this may also enhance the visibility of the illuminable instruction panel 3110 when the light source 3204 is activated. In one example, if the illuminable instruction panel 3110 is substantially translucent, then the illuminable instruction panel 3110 may appear to assume the color of the light source 3204 when the light source 3204 is activated. In one specific example, if the light source 3204 has a red color, and the illuminable instruction panel 3110 is substantially translucent, then the illuminable instruction panel 3110 may appear to have a red color when the light source 3204 is activated to illuminate the illuminable instruction panel 3110. As should be appreciated, a variety of designs may be employed for a displayed instruction. For example, in some cases, only the letters in the words “remove cap” may be translucent and may be illuminated by the underlying light source 3204. In other examples, the words “remove cap” could be colored (e.g., colored black or yellow), while the remainder of the illuminable instruction panel 3110 may be translucent and may be illuminated by the underlying light source 3204. In this example, the remove cap instruction would still be considered to be illuminated even if the letters in the words “remove cap” were not necessarily themselves illuminated. Any, or all, of the above, or other, examples of illuminable instructions may be similarly employed for alternative instructions, such as instructions to shake the device, to press down the device for injection, and the like.

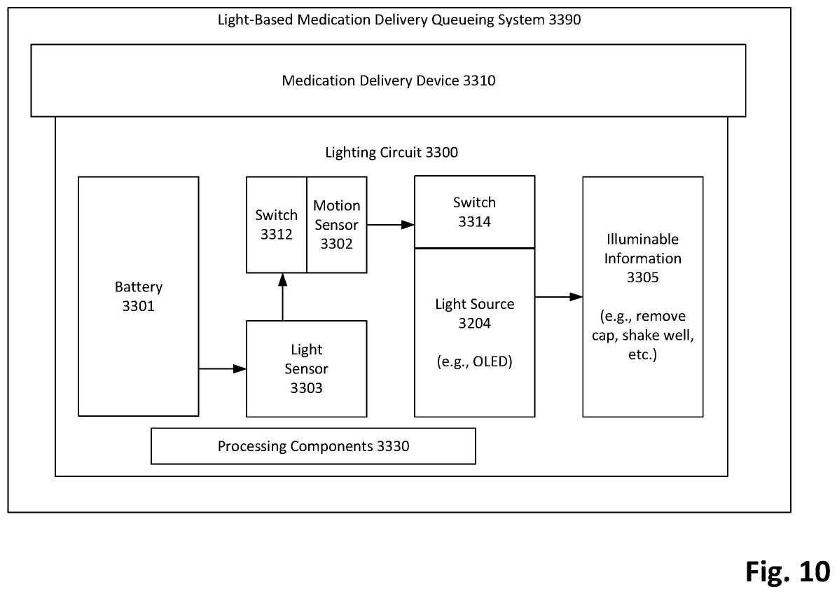

Referring now to FIG. 10, an example of a light-based medication delivery cueing system 3390 will now be described in detail. As shown, the light-based medication delivery cueing system 3390 includes a lighting circuit 3300 and a medication delivery device 3310, such as device 300 of FIG. 7.

The lighting circuit 3300 may be integrated with the medication delivery device 3310. The term integrated with, as used herein, means that the lighting circuit 3300 is included within the medication delivery device 3310 or is otherwise directly, or indirectly (e.g., via one or more connecting or attaching components), physically connected, or attached, to (e.g., via an adhesive, etc.) the medication delivery device 3310, before and/or at the time that the medication delivery device is used by a patient (and/or by another user on the patient's behalf). Thus, battery 3301, motion sensor 3302, light sensor 3303, processing components 3330, switch 3312, light source 3204, switch 3314, and illuminable information 3305 may be integrated with the medication delivery device 3310. In some examples, the lighting circuit 3300 may be included in one or more thin and flexible adhesive labels that are attachable to, and/or attached to, the medication delivery device 3310. Thus, in some examples, any, or all, of battery 3301, motion sensor 3302, light sensor 3303, processing components 3330, switch 3312, light source 3204, switch 3314, and illuminable information 3305 may be included in one or more thin and flexible adhesive label that are attachable to, and/or attached to, the medication delivery device 3310. Additionally, in some examples, the lighting circuit 3300 may be included within the medication delivery device 3310. For example, the lighting circuit 3300 may be embedded into one or more other components of the medication delivery device 3310, such as by being molded into plastic and/or other materials of which those components may be comprised. Thus, in some examples, any, or all, of battery 3301, motion sensor 3302, light sensor 3303, processing components 3330, switch 3312, light source 3204, switch 3314, and illuminable information 3305 may be included within the medication delivery device 3310.

The lighting circuit 3300 includes a light sensor 3303, which is configured to detect the presence of light exterior to the medication delivery device 3310. In one specific example, light sensor 3303 (and optionally other light sensors described herein) may be a semiconductor-based sensor, such as may convert light into current, for example via a junction between different types of semiconductor materials. The light sensor 3303 is powered by battery 3301. In some examples, prior to being used by a patient or other user, the medication delivery device 3310 may be stored in a storage component, such as storage carton 700 of FIG. 5. In order to use the medication delivery device 3310, the patient may first need to remove the medication delivery device 3310 from the storage component. In some examples, while in the storage component, the medication delivery device 3310 may not be exposed to any light. Thus, in some examples, when the light sensor 3303 detects light, this may correspond to a removal of the medication delivery device 3310 from the storage component.

The lighting circuit 3300 also includes motion sensor 3302. The motion sensor 3302 is configured to detect motion of the medication delivery device 3310, such as caused by a user's movement of the medication delivery device 3310. This may occur, for example, when a user picks up the medication delivery device 3310, such as to inject or otherwise obtain the medication from the medication delivery device 3310. In some examples, the motion sensor 3302 (and optionally other motion sensors described herein) may include an accelerometer, such as to detect acceleration of the medication delivery device 3310. In one specific example, motion sensor 3302 (as well as other motion sensors described herein) may be an accelerometer that measures acceleration forces in a plurality of directions (e.g., two or three axes) and reports these measurements to processing components 3330. The combination of the removal of the medication delivery device 3310 from a storage component (as detected by light sensor 3303) and subsequent motion of the medication delivery device 3310 (as detected by motion sensor 3302) may indicate that a user is ready to dispense a medication from the medication delivery device.

In some examples, the motion sensor 3302 may initially be in a sleep state in which it is not powered. The motion sensor 3302 may remain unpowered until the light is detected by the light sensor 3303. When light is detected by the light sensor 3303, the motion sensor 3302 may then be powered-on by receiving power from battery 3301. In this manner, power of the battery 3301 may be conserved. Additionally, this may prevent the motion sensor 3302 from detecting motion of the medication delivery device 3310 while the medication delivery device 3310 is still inside the storage component, which would not indicate that the user is ready to use the medication delivery device. Switch 3312 is an electrical switch that may control the flow of power from the battery 3301 to the motion sensor 3302. The switch 3312 may be initially in an open state (which does not allow power to be provided to the motion sensor 3302) and then, upon detection of the light, moved to a closed state (which does allow power to be provided to the motion sensor 3302). In some examples, the light sensor 3303 may, upon detection of the light, provide an indication (e.g., signal) of the detected light to processing components 3330, which, in turn, may provide an indication (e.g., signal) to close the switch 3312 and allow power to flow to the motion sensor 3302. In some examples, processing components 3330 may be wholly or partially integrated with light sensor 3303 or may be separate components. In some examples, processing components 3330 (as well as other processing components described herein) may be components that are programmed, or otherwise instructed, to perform the operations that they execute as described herein, and that may be included in one or more integrated circuit (IC) chips and/or other IC components.

As described above, the combination of the removal of the medication delivery device 3310 from a storage component (as detected by light sensor 3303) and subsequent motion of the medication delivery device 3310 (as detected by motion sensor 3302) may indicate that a user is ready to deliver a medication from the medication delivery device. Thus, in this example, the light source 3204 may be activated after the detection of both the light and the motion. Light source 3204 may be activated by being changed to one or more active states, such as to start emitting light (e.g., by being powered-on), to start flashing, to change color (e.g., to change to a green color or another designated color associated with an active state), and the like. When activated, the light source 3204 may illuminate the illuminable information 3305.

In the example of FIG. 10, light source 3204 may be activated by being caused to emit light, such as by being powered-on. Switch 3314 is an electrical switch that may control the flow of power from the battery 3301 to the light source 3204. The switch 3314 may be initially in an open state (which does not allow power to be provided to the light source 3204) and then, upon detection of the motion, moved to a closed state (which does allow power to be provided to the light source 3204). In some examples, the motion sensor 3302 may, upon detection of the motion, provide an indication (e.g., signal) of the detected motion to processing components 3330, which, in turn, may provide an indication (e.g., signal) to close the switch 3314 and allow power to flow to the light source 3204. In some examples, processing components 3330 may be wholly or partially integrated with motion sensor 3302 or may be separate components. In this manner, the light source 3204 may be activated based at least in part on the detected motion. Additionally, because the motion sensor 3302 is powered based on detection of the light, it is noted that both light detection and motion detection may be required to activate the light source 3204. In this manner, the light source 3204 may be activated based at least in part on both the detected light and the detected motion (even though the light sensor 3303 may not directly activate the light source 3204). As discussed above, keeping the motion sensor 3302 in a sleep state prior to detection of the light (e.g., while in the storage component) may help to prevent the battery 3301 from running out of power.

In some examples, any, or all, of the lighting circuit components (e.g., motion sensor 3302, light sensor 3303, light source 3204, switch 3312, switch 3314, processing components 3330, battery 3301) of the lighting circuit 3300 (as well as other lighting circuits described herein) may be implemented, in whole or in part, using one or more integrated circuit (IC) components, such as one or IC chips. The integrated circuit components may be connected to one or more circuit boards, such as a printed circuit board and/or flexible circuit board. The components may communicate and/or interact with one another via one or more electrical connections, for example via the one or more circuit boards. Any, or all, of the lighting circuit components of the lighting circuit 3300 shown in FIG. 10 (as well as other lighting circuits described herein) may optionally include one or more processing components (e.g., integrated with or separate from processing components 3330) and one or more memory components. The memory components can be volatile (such as some types of RAM), non-volatile (such as ROM, flash memory, etc.), or a combination thereof. Additionally, in some examples, any or all of the lighting circuits described herein may be wholly or partially combined with one another.

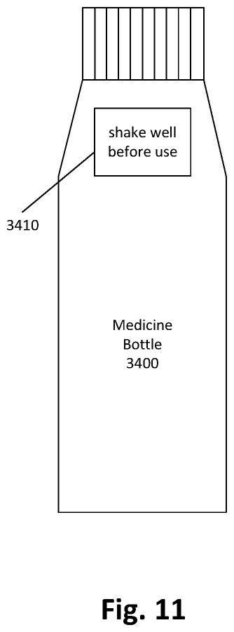

In some examples, activation of light source 3204 may be a visual cue to perform a medication delivery instruction, such as a cap removal instruction or a device shaking instruction. In some examples, the illuminable information 3305 may include text corresponding to these instructions, such as the words “remove cap” or the words “shake well”. In one specific example, the medication delivery device 3310 may be device 300 of FIG. 7, and the illuminable information 3305 may be illuminable instruction panel 3110 of FIG. 7, which displays the words “remove cap”. In another specific example, the medication delivery device 3310 may be a medicine bottle that stores a medication that is in a liquid form, and the illuminable information 3305 may display the words “shake well before use”. Referring now to FIG. 11, an example is shown in which a medicine bottle 3400 includes an illuminable instruction panel 3410. In the example of FIG. 11, the illuminable instruction panel 3410 displays the words “shake well before use”. Thus, medicine bottle 3400 is another example of medication delivery device 3310. The medicine bottle 3400 may also include lighting circuit 3300 of FIG. 10. The illuminable instruction panel 3410 is another example of illuminable information 3305. The illuminable instruction panel 3410 may be illuminated by light source 3204 based on detected light and detected motion as described above with respect to lighting circuit 3300.

In some examples, it may be desirable to cue the user to perform multiple medication delivery instructions in a designated sequence. One specific example of this relates to a tamper-resistant blister pack, such as for dispensing medication pills, caplets, etc. Specifically, in some examples, to obtain a medication from this type of blister pack, a user may be required to first press down on, and hold, a release tab. The user may then pull out the desired medication from the blister pack only while pressing down on the release tab, thereby making it difficult for someone to improperly access the medication. In some examples, the light and motion sensing techniques described above may be employed to cue the user to perform a first instruction, such as for the user to push down and hold the release tab. Then, only after the first instruction is performed, the user may be cued to perform a second instruction, such as to pull out the medication from the blister pack.

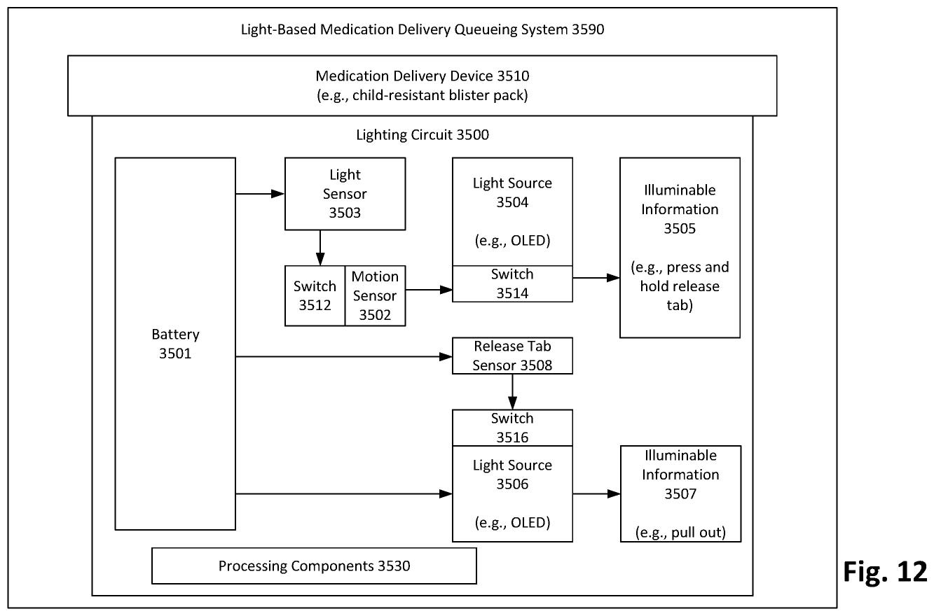

Referring now to FIG. 12, another example is shown of a light-based medication delivery cueing system 3590. The light-based medication delivery cueing system 3590 includes a lighting circuit 3500 and a medication delivery device 3510, such as the tamper-resistant blister pack described above.

The lighting circuit 3500 may be integrated with the medication delivery device 3510. The term integrated with, as used herein, means that the lighting circuit 3500 is included within the medication delivery device 3510 or is otherwise directly, or indirectly (e.g., via one or more connecting or attaching components), physically connected, or attached, to (e.g., via an adhesive, etc.) the medication delivery device 3510, before and/or at the time that the medication delivery device is used by a patient (and/or by another user on the patient's behalf). Thus, battery 3501, motion sensor 3502, light sensor 3503, processing components 3530, switch 3512, switch 3514, switch 3516, light source 3504, light source 3506, release tab sensor 3508, illuminable information 3505 and illuminable information 3507 may be integrated with the medication delivery device 3510. In some examples, the lighting circuit 3500 may be included in one or more thin and flexible adhesive labels that are attachable to, and/or attached to, the medication delivery device 3510. Thus, in some examples, any, or all, of battery 3501, motion sensor 3502, light sensor 3503, processing components 3530, switch 3512, switch 3514, switch 3516, light source 3504, light source 3506, release tab sensor 3508, illuminable information 3505 and illuminable information 3507 may be included in one or more thin and flexible adhesive label that are attachable to, and/or attached to, the medication delivery device 3510. Additionally, in some examples, the lighting circuit 3500 may be included within the medication delivery device 3510. For example, the lighting circuit 3500 may be embedded into one or more other components of the medication delivery device 3510, such as by being molded into plastic and/or other materials of which those components may be comprised. Thus, in some examples, any, or all, of battery 3501, motion sensor 3502, light sensor 3503, processing components 3530, switch 3512, switch 3514, switch 3516, light source 3504, light source 3506, release tab sensor 3508, illuminable information 3505 and illuminable information 3507 may be included within the medication delivery device 3510.

In the example of FIG. 12, light sensor 3503 may operate in the same manner as light sensor 3303 of FIG. 10, such as to detect light corresponding to removal of medication delivery device 3510 from a storage component and to cause power to be provided to motion sensor 3502, from battery 3501, based on the detected light. Switch 3512 is an electrical switch that may be initially open (which does not allow power to be provided to the motion sensor 3502) and then closed, based on detection of the light, to allow power to be provided to motion sensor 3502. Additionally, motion sensor 3502 may operate in the same manner as motion sensor 3302 of FIG. 10, such as to detect motion of the medication delivery device 3510 and to activate light source 3504 (e.g., an OLED, OLED display, LED, etc.) based on the detected motion. In the example of FIG. 12, light source 3504 may be activated by being caused to emit light, such as by being powered-on. Switch 3514 is an electrical switch that may be initially open (which does not allow power to be provided to the light source 3504) and then closed, based on detection of the motion, to allow power to be provided to light source 3504. Activation of light source 3504 may be a visual cue to perform a first medication delivery instruction, such as an instruction to press down, and hold, the release tab of the tamper-resistant blister pack. Light source 3504 may illuminate illuminable information 3505, such as may include text (e.g., the words “press down and hold”) and/or symbols (e.g., an arrow pointing to the release tab).

In this example, lighting circuit 3500 further includes a release tab sensor 3508, such as a touch sensor and/or pressure sensor, that is configured to detect when the user has pressed the release tab. In some examples, the light source 3504 may be powered-off (e.g., by opening switch 3514) after either expiration of a designated time period (e.g., five seconds) or the user pressing the release tab (e.g., as detected by the release tab sensor 3508). For example, upon detecting the pressing of the release tab, the release tab sensor 3508 may cause switch 3514 to be moved from a closed state (which does allow power to be provided to the light source 3504) back to an open state (which does not allow power to be provided to the light source 3504).

Additionally, upon detection of the user pressing the release tab, the release tab sensor 3508 may also cause light source 3506 (e.g., an OLED, OLED display, LED, etc.) to be activated. In the example of FIG. 12, light source 3506 may be activated by being caused to emit light, such as by being powered-on. Switch 3516 is an electrical switch that may control the flow of power from the battery 3501 to the light source 3506. The switch 3516 may be initially in an open state (which does not allow power to be provided to the light source 3506) and then, upon detection of pressing down of the release tab, moved to a closed state (which does allow power to be provided to the light source 3506). In some examples, the release tab sensor 3508 may, upon detection of the pressing down of the release tab, provide an indication (e.g., signal) of the detected pressing down of the release tab to processing components 3530, which, in turn, may provide an indication (e.g., signal) to close the switch 3516 and allow power to flow to the light source 3506. Activation of light source 3506 may be a visual cue to perform a second medication delivery instruction, such as an instruction to pull the medication out from the tamper-resistant blister pack. Upon being activated, the light source 3506 may cause illumination of illuminable information 3507, such as may include text and/or symbols instructing the user to pull the medication out from the tamper-resistant blister pack.

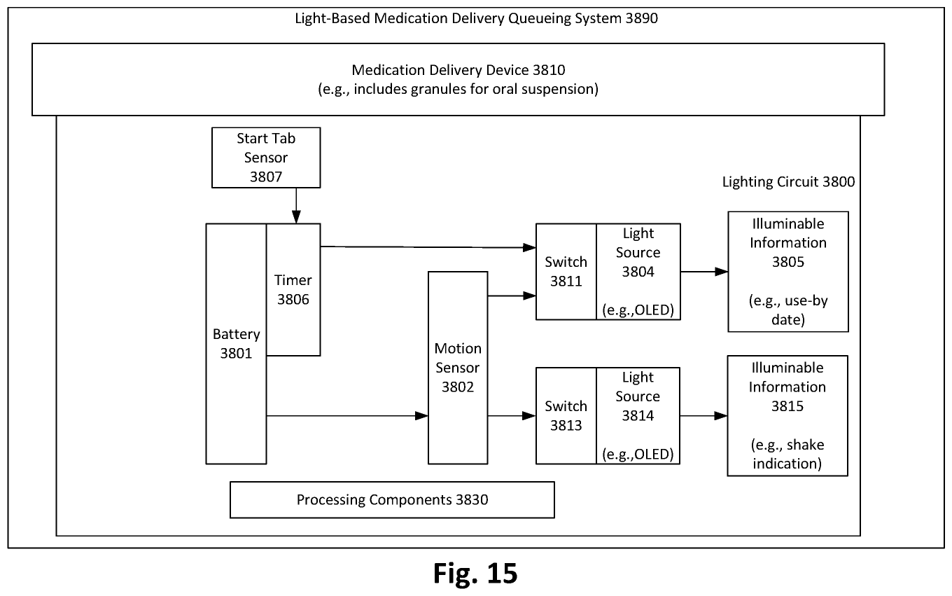

It is noted that FIG. 12 and the tamper-resistant blister pack are merely one example of a scenario in which it may be desirable to cue the user to perform multiple medication delivery instructions. For example, FIG. 15, which is described below, provides an example in which visual indications related to a use-by date and shaking of a device may be provided. In yet other examples, different illuminable information could be displayed by different light sources in response to different motions/motion sequences being detected by sensors. For example, “remove cap,” then “place on skin,” then “press here” or the like. In some examples, a motion sensor may be employed adjacent to the device cap to detect removal of the cap, such as a motion of the user's hand removing the cap from the device. Detection of the cap removal motion could then trigger a light source that illuminates instructions to place the device on the user's skin. Detection of the cap removal motion may also trigger another motion sensor that detects a motion associated with placing a device on the user's skin, such as a downward or horizontal motion (depending on the body part being injected). Detection of the placement of the device on the user's skin could then trigger a light source that illuminates instructions to press down on the device. Other alternative combinations using different sensors (e.g., light sensors, force sensors, etc.) and different instructions may also be employed.

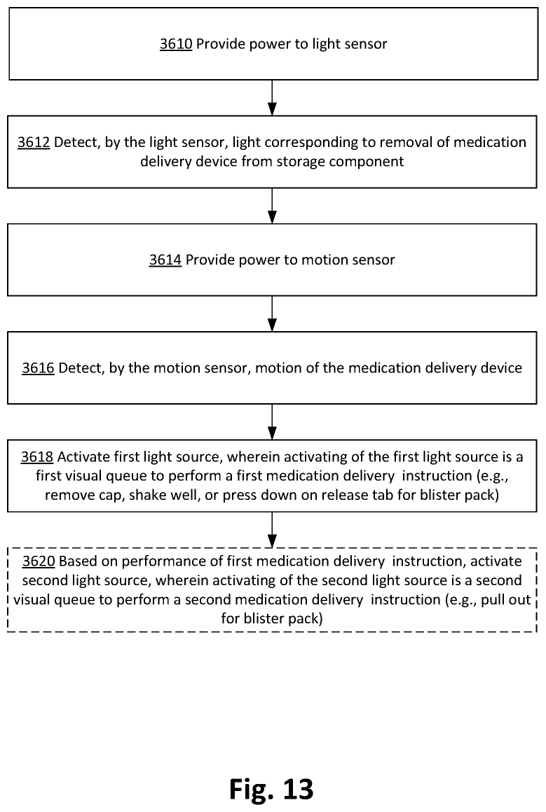

Referring now to FIG. 13, a flowchart for an example light-based medication delivery cueing process will now be described in detail. In some examples, the process of FIG. 13 may be performed by lighting circuit 3300 of FIG. 10 and/or lighting circuit 3500 of FIG. 12. At operation 3610, power is provided to a light sensor. For example, as shown in FIG. 10, power is provided to light sensor 3303 from battery 3301. Providing power to a component may also be referred to as powering the component. At operation 3612, light corresponding to a removal of the medication delivery device from a storage component is detected by the light sensor. For example, as described above, prior to being used by a patient or other user, a medication delivery device may be stored in a storage component, such as storage carton 700 of FIG. 5. While in the storage component, the medication delivery device may not be exposed to any light (or very little light). Thus, in some examples, when the light sensor 3303 detects light, this may correspond to a removal of the medication delivery device 3310 from the storage component. In some examples, the detection of light at operation 3612 may include detecting at least a threshold amount of light, such as an amount of light that is greater than an amount of light to which the medication delivery device is exposed in the storage component. In some examples, the light sensor 3303 may provide indications, to the processing components 3330, of amounts of light that are detected by the light sensor 3303 over time. The processing components 3330 may then compare a detected amount of light to the threshold amount of light, and the processing components 3930 may cause power to be provided to the motion sensor based of the detected amount of light meeting or exceeding the threshold.

At operation 3614, power is provided to the motion sensor. Power may be provided to the motion sensor (at operation 3614) based at least in part on the detecting of the light (at operation 3612), such as based on detecting of a threshold amount of light. As described above, a switch 3312 may control the flow of power from the battery 3301 to the motion sensor 3302. The switch 3312 may be initially in an open state (which does not allow power to be provided to the motion sensor 3302) and then, upon detection of the light, moved to a closed state (which does allow power to be provided to the motion sensor 3302). In some examples, the light sensor 3303 may, upon detection of the light, provide an indication (e.g., signal) of the detected light to processing components 3330, which, in turn, may provide an indication (e.g., signal) to close the switch 3312 and allow power to flow to the motion sensor 3302. Thus, as described above, the motion sensor 3302 may be switched from a sleep state (which conserves battery power) to a powered state when the device is removed from a storage component.

At operation 3616, motion of the medication delivery device is detected by the motion sensor. The detected motion may correspond to a user's movement of the medication delivery device. In some examples, the detection of motion at operation 3616 may include detecting at least a threshold amount of motion (and/or threshold amounts of motion in one or more specified axes or directions). In some examples, the motion sensor 3302 may provide indications, to the processing components 3330, of amounts of motion that are detected by the motion sensor 3302 over time. The processing components 3330 may then compare a detected amount of motion to the threshold amount of motion, and the processing components 3930 may cause the first light source to be activated based of the detected amount of motion meeting or exceeding the threshold. At operation 3618, a first light source is activated. The first light source may be activated by being changed to one or more active states, such as to start emitting light (e.g., by being powered-on), to start flashing, to change color (e.g., to change to a green color or another designated color associated with an active state), and the like. The first light source may be activated (at operation 3618) based at least in part on the detecting of the motion (at operation 3616), such as based on detecting of a threshold amount of motion. As also described above, because the motion sensor 3302 may be powered based on detection of the light, the first light source may be activated based at least in part on detection of the light and detection of the motion. In the example of FIG. 10, light source 3204 may be activated by being caused to emit light, such as by being powered-on. As described above, a switch 3314 may control the flow of power from the battery 3301 to the light source 3204. The switch 3314 may be initially in an open state (which does not allow power to be provided to the light source 3204) and then, upon detection of the motion, moved to a closed state (which does allow power to be provided to the light source 3204). In some examples, the motion sensor 3302 may, upon detection of the motion, provide an indication (e.g., signal) of the detected motion to processing components 3330, which, in turn, may provide an indication (e.g., signal) to close the switch 3314 and allow power to flow to the light source 3204. The activating of the first light source is a first visual cue to perform a first medication delivery instruction (e.g., a remove cap instruction as shown in FIG. 10 or a shake well instruction as shown in FIG. 11). In some examples, the first light source may illuminate text and/or a symbol (e.g., an arrow, etc.).

In some examples, such as shown in FIG. 12, a user may be cued to perform a second medication delivery instruction. The user may be cued to perform this second medication delivery instruction based on the user performing the first medication delivery instruction. For example, for a tamper-resistant blister pack, the first instruction could be an instruction to press down and hold on a release tab and the second instruction could be an instruction to pull out the medication from the blister pack. In these and other examples, an additional operation (operation 3620) may be performed. Specifically, at operation 3620, based on performance of the first medication delivery instruction (e.g., pressing down and holding a release tab), a second light source is activated. The activating of the second light source is a second visual cue to perform a second medication delivery instruction (e.g., pull out medication from blister pack). As shown in FIG. 12, an additional sensor, such as an additional light sensor, touch sensor and/or motion sensor, may be employed to detect when the first medication delivery instruction is performed. As a specific example, release tab sensor 3508, such as a touch sensor, is an additional sensor that may be employed to determine when the release tab is pressed, which may cause activation of the second light source.

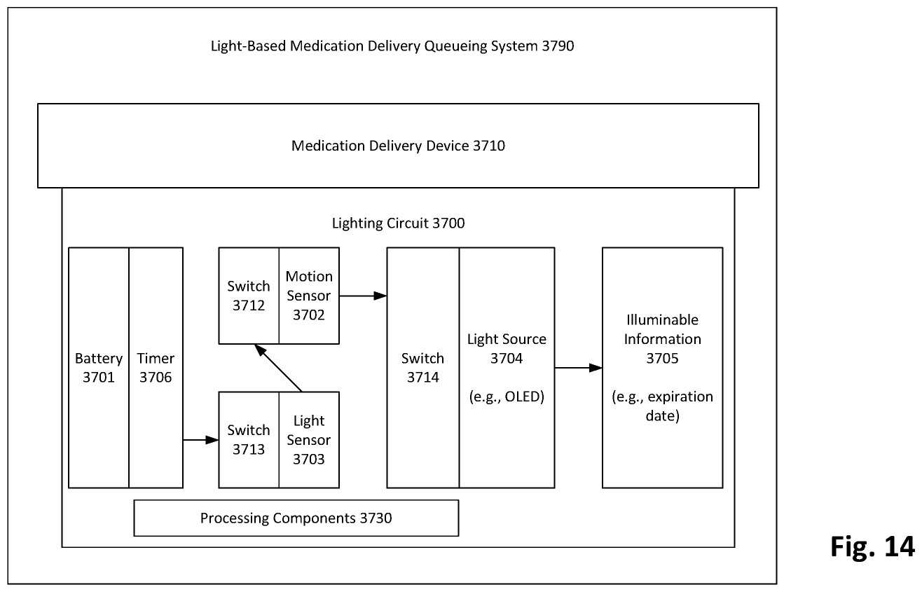

Referring now to FIG. 14, an example of a light-based medication delivery cueing system 3790 for illuminating an expiration date will now be described in detail. As shown, light-based medication delivery cueing system 3790 includes medication delivery device 3710 and lighting circuit 3700.

The lighting circuit 3700 may be integrated with the medication delivery device 3710. The term integrated with, as used herein, means that the lighting circuit 3700 is included within the medication delivery device 3710 or is otherwise directly, or indirectly (e.g., via one or more connecting or attaching components), physically connected, or attached, to (e.g., via an adhesive, etc.) the medication delivery device 3710, before and/or at the time that the medication delivery device is used by a patient (and/or by another user on the patient's behalf). Thus, battery 3701, timer 3706, motion sensor 3702, light sensor 3703, processing components 3730, switch 3712, switch 3713, switch 3714, light source 3704, and illuminable information 3705 may be integrated with the medication delivery device 3710. In some examples, the lighting circuit 3700 may be included in one or more thin and flexible adhesive labels that are attachable to, and/or attached to, the medication delivery device 3710. Thus, in some examples, any, or all, of battery 3701, timer 3706, motion sensor 3702, light sensor 3703, processing components 3730, switch 3712, switch 3713, switch 3714, light source 3704, and illuminable information 3705 may be included in one or more thin and flexible adhesive label that are attachable to, and/or attached to, the medication delivery device 3710. Additionally, in some examples, the lighting circuit 3700 may be included within the medication delivery device 3710. For example, the lighting circuit 3700 may be embedded into one or more other components of the medication delivery device 3710, such as by being molded into plastic and/or other materials of which those components may be comprised. Thus, in some examples, any, or all, of battery 3701, timer 3706, motion sensor 3702, light sensor 3703, processing components 3730, switch 3712, switch 3713, switch 3714, light source 3704, and illuminable information 3705 may be included within the medication delivery device 3710.

Lighting circuit 3700 includes a timer 3706, which is configured to determine an occurrence of a designated date (e.g., a medication expiration date). The timer 3706 may be an integrated circuit (IC) chip that is programmed to determine when the designated date has been reached. In one specific example, the timer 3706 may be programmed when a medication is packaged for distribution. The timer 3706 may be powered by battery 3701.

The lighting circuit 3700 further includes light sensor 3703 and motion sensor 3702, which may both also be powered by battery 3701. Light sensor 3703 may detect light corresponding to a removal of the medication delivery device 3710 from a storage component. Motion sensor 3702 may detect motion of the medication delivery device. In this example, both light sensor 3703 and motion sensor 3702 may initially be in a sleep state in which they are not powered. The light sensor 3703 may remain unpowered until the occurrence of the designated date (e.g., medication expiration date) is detected by the timer 3706. Thus, the light sensor 3703 is powered based on a detection of the occurrence of the designated date. When the occurrence of the designated date is detected by the timer 3706, the light sensor 3703 may then be changed to a powered state, in which light sensor 3703 receives power from battery 3701. Switch 3713 may control the flow of power from the battery 3701 to the light sensor 3703. The switch 3713 may be initially in an open state (which does not allow power to be provided to the light sensor 3703) and then, upon detection of the occurrence of the designated date, moved to a closed state (which does allow power to be provided to the light sensor 3703). In some examples, the timer 3706 may, upon detection of the occurrence of the designated date, provide an indication of the occurrence of the designated date to processing components 3730, which, in turn, may provide a signal to close the switch 3713 and allow power to flow to the light sensor 3703. The light sensor 3703 detects light corresponding to a removal of the medication delivery device 3710 from a storage component. The motion sensor 3702 may then be powered based at least in part on the detection, by light sensor 3703, of the light corresponding to the removal of the medication delivery device 3710 from the storage component. Switch 3712 may control the flow of power from the battery 3701 to the motion sensor 3702. The switch 3712 may be initially in an open state (which does not allow power to be provided to the motion sensor 3702) and then, upon detection of the light, moved to a closed state (which does allow power to be provided to the motion sensor 3702). In some examples, the light sensor 3703 may, upon detection of the light, provide an indication of the detection of the light to processing components 3730, which, in turn, may provide a signal to close the switch 3712 and allow power to flow to the motion sensor 3702.

The lighting circuit 3700 further includes light source 3704, such as an OLED, OLED display, LED, etc. Activation of the light source 3704, may cause illumination of illuminable information 3705, which is medication delivery information for delivering a medication. Thus, activation of the light source 3704 may cause illumination of medication delivery information (e.g., illuminable information 3705). The medication delivery information may relate to an expiration date of a medication included in the medication delivery device 3710. For example, the medication delivery information may include the expiration date, the word “expired” or the like, and/or a corresponding symbol (e.g., an hourglass, etc.). Thus, activation of light source 3704 may be a visual cue for the user to perform a medication delivery instruction, such as to check the expiration date of the medication. In this example, the light source 3704 may be activated based at least in part on detection of the motion of the medication delivery device by motion sensor 3702. The light source 3704 may be activated by being changed to one or more active states, such as to start emitting light (e.g., by being powered-on), to start flashing, to change color (e.g., to change to a green color or another designated color associated with an active state), and the like. In the example of FIG. 14, light source 3704 may be activated by being caused to emit light, such as by being powered-on. Switch 3714 may control the flow of power from the battery 3701 to the light source 3704. Switch 3714 may be initially in an open state (which does not allow power to be provided to the light source 3704) and then, upon detection of the motion, moved to a closed state (which does allow power to be provided to the light source 3704). The motion sensor 3702 may, upon detection of the motion, provide an indication of the detection of the motion to processing components 3730, which, in turn, may provide a signal to close the switch 3714. In an alternative embodiment, both the motion sensor 3702 and the light sensor 3703 may become powered when the occurrence of the designated date is detected by the timer 3706. In this embodiment, the light source 3704 may be powered based on detection of the light (corresponding to the removal of the medication delivery device 3710 from the storage component) by the light sensor 3703 and/or the detection of the motion (corresponding to the motion of the medication delivery device) by the motion sensor 3702.

In the example of FIG. 14, the motion sensor 3702 is powered based on detection of the light by the light sensor 3703. For this reason, the light source 3704 may be activated based on detection of the light and detection of the motion, for example because detection of the light is necessary to power motion sensor 3702. In some examples, the lighting circuit 3700 may not include motion sensor 3702, and the light source 3704 may be activated directly in response to detection of the light. Thus, it is noted that light source 3704 may be activated based at least in part on detection of the light. For example, the light source 3704 may be activated directly in response to detection of the light (e.g., when there is no motion sensor). Additionally, the light source 3704 may also be activated based at least in part on detection of the light, for example when detection of the light causes power to be applied to the motion sensor 3703 as shown in FIG. 14.

In this example, the timer 3706 prevents the light sensor 3703 and motion sensor 3702 from being powered until the designated date (e.g., expiration date) occurs. This prevents the illuminable information 3705 from being illuminated prior to occurrence of the designated date (e.g., expiration date). This is advantageous, for example, because it conserves battery power prior to the designated date, while also allowing the illuminable information 3705 to be illuminated once the designated date has occurred. It is noted that, while FIG. 14, shows an example including both a motion sensor 3702 and a light sensor 3703, some other examples may include only a single sensor, such as a motion sensor (with no light sensor) or a light sensor (with no motion sensor). In these single sensor examples, the occurrence of the designated date may trigger power to be applied to the single sensor, and the detecting of light or motion by the single sensor may trigger activation of the light source 3704.

Referring now to FIG. 15, another example is shown of a light-based medication delivery cueing system 3890. The light-based medication delivery cueing system 3890 includes a lighting circuit 3800 and a medication delivery device 3810, such as may include granules for oral suspension or a liquid emulsion.

The lighting circuit 3800 may be integrated with the medication delivery device 3810. The term integrated with, as used herein, means that the lighting circuit 3800 is included within the medication delivery device 3810 or is otherwise directly, or indirectly (e.g., via one or more connecting or attaching components), physically connected, or attached, to (e.g., via an adhesive, etc.) the medication delivery device 3810, before and/or at the time that the medication delivery device is used by a patient (and/or by another user on the patient's behalf). Thus, battery 3801, start tab sensor 3807, timer 3806, motion sensor 3802, processing components 3830, switch 3811, switch 3813, light source 3804, light source 3814, illuminable information 3805 and illuminable information 3815 may be integrated with the medication delivery device 3810. In some examples, the lighting circuit 3800 may be included in one or more thin and flexible adhesive labels that are attachable to, and/or attached to, the medication delivery device 3810. Thus, in some examples, any, or all, of battery 3801, start tab sensor 3807, timer 3806, motion sensor 3802, processing components 3830, switch 3811, switch 3813, light source 3804, light source 3814, illuminable information 3805 and illuminable information 3815 may be included in one or more thin and flexible adhesive label that are attachable to, and/or attached to, the medication delivery device 3810. Additionally, in some examples, the lighting circuit 3800 may be included within the medication delivery device 3810. For example, the lighting circuit 3800 may be embedded into one or more other components of the medication delivery device 3810, such as by being molded into plastic and/or other materials of which those components may be comprised. Thus, in some examples, any, or all, of battery 3801, start tab sensor 3807, timer 3806, motion sensor 3802, processing components 3830, switch 3811, switch 3813, light source 3804, light source 3814, illuminable information 3805 and illuminable information 3815 may be included within the medication delivery device 3810.

In some examples, a medication in the form of granules for oral suspension may be reconstituted (e.g., by a pharmacist) or a liquid emulsion. The medication may be required to be used by a use-by date, such as may be based on a date when the medication is reconstituted. In some examples, the pharmacist may write the use-by date on the medication delivery device. The medication may also be required to be shaken by a patient before use.

In the example of FIG. 15, upon reconstituting the medication, a pharmacist may activate the start tab sensor 3807 (e.g., a touch and/or pressure sensor), such as by pressing or pulling on a start tab. The activation of start tab sensor 3807 may energize the lighting circuit 3800. The lighting circuit includes a timer 3806, which may be configured to detect an expiration of a designated time period, such as may expire on the use-by date of the medication. Specifically, an indication of the activation of start tab sensor 3807 may be sent to processing components 3830, which, in turn, may cause a timer 3806 to start counting down the designated time period. Light source 3804 may be activated based at least in part on detection of the expiration of the designated time period. Light source 3804 may be activated by being changed to one or more active states, such as to start emitting light (e.g., by being powered-on), to start flashing, to change color (e.g., to change to a green color or another designated color associated with an active state), and the like. In the example of FIG. 15, light source 3804 may be activated by being caused to emit light, such as by being powered-on. In particular, upon expiration of the designated time period, the timer 3806 may cause the battery 3801 to provide power to light source 3804 (e.g., an OLED, OLED display, LED, etc.). Switch 3811 may control the flow of power from battery 3801 to light source 3804. Switch 3811 may initially be in an open position (which does not allow power to be provided to the light source 3804) and then, upon expiration of the designated time period, moved to a closed position (which does allow power to be provided to the light source 3804). Timer 3806 may provide an indication of the expiration of the designated time period to processing components 3830, which, in turn, may provide a signal to close switch 3811. Activation of the light source 3804 may cause illumination of illuminable information 3805, which is first medication delivery information. Thus, activation of the light source 3804 may cause illumination of first medication delivery information (e.g., illuminable information 3805). The first medication delivery information may include information related to the use-by date of a medication included in medication delivery device 3810, such as the words “use-by” positioned adjacent to the use-by date that may be written by a pharmacist. In some examples, when the designated time period expires, the light source 3804 may illuminate for a set time period (e.g., ten minutes) and may pulse. Thus, activation of light source 3804 may be a visual cue for the user to perform a medication delivery instruction, such as to check the use-by date of the medication.

Additionally, lighting circuit 3800 includes a motion sensor 3802 that detects motion of the medication delivery device 3810. In some examples, the motion sensor 3802 may include an accelerometer that may be configured to detect a threshold motion of the medication delivery device 3810, such as a threshold motion corresponding to shaking of the medication delivery device by a user. In some examples, the motion sensor 3802 may provide indications, to the processing components 3830, of amounts of motion that are detected by the motion sensor 3802 over time. The processing components 3830 may then compare a detected amount of motion to the threshold amount of motion, and the processing components 3830 may determine when the detected amount of motion meets or exceeds the threshold. When this threshold motion is detected prior to expiration of the designated use-by time period, a count-down of another time period for shaking of medication delivery device 3810 may be started. The time period for shaking may also be measured by timer 3806 (or an additional timer not shown in FIG. 15). Upon expiration of this shaking time period, light source 3814 (e.g., an OLED, OLED display, LED, etc.) may be activated to provide an indication to stop shaking the medication delivery device 3810. Thus, the light source 3814 may be activated based at least in part on detection of motion of the medication delivery device. Light source 3814 may be activated by being changed to one or more active states, such as to start emitting light (e.g., by being powered-on), to start flashing, to change color (e.g., to change to a green color or another designated color associated with an active state), and the like. In the example of FIG. 15, light source 3814 may be activated by being caused to emit light, such as by being powered-on. Switch 3813 may control the flow of power from battery 3801 to light source 3814. Switch 3813 may initially be in an open position (which does not allow power to be provided to the light source 3814) and then, upon completion of the shaking time period, moved to a closed position (which does allow power to be provided to the light source 3814). Timer 3806 (or an additional timer not shown in FIG. 15) may provide an indication of the completion of the shaking time period to processing components 3830, which, in turn, may provide a signal to close switch 3813. In some other examples, motion sensor 3802 (as well as other motion sensors described herein) may be an accelerometer that measures acceleration forces in a plurality of directions (e.g., two or three axes) and reports these measurements to processing components 3830. The processing components 3830 may then employ an algorithm to determine when enough force has been applied in plurality of directions (e.g., two or three axes) to equate to a successful shake.

Activation of the light source 3814 may cause illumination of illuminable information 3815, which is second medication delivery information. Thus, activation of the light source 3814 may cause illumination of second medication delivery information (e.g., illuminable information 3815). The second medication delivery information may include an instruction related to shaking of the medication delivery device 3810, such as an illuminable symbol adjacent to words “stop shaking when this symbol stays illuminated”. This is a visual cue to the patient that the medication has been shaken for the necessary amount of time. Thus, activation of light source 3814 may be a visual cue for the user to perform a medication delivery instruction, such as to stop shaking the medication after the necessary amount of time.

After the designated time period corresponding to the use-by date has expired, the medication should no longer be used. Thus, after expiration of the designated use-by time period, the light source 3814 may not be activated. Instead, when the motion sensor 3802 detects motion of the medication delivery device 3810 after the expiration of the designated use-by time period, the light source 3804 may be activated. Activation of light source 3804 may cause illumination of illuminable information 3805, which, as described above, may include information associated with the use-by date, such as the words “use-by” positioned adjacent to the use-by date that may be written by a pharmacist. This may help to ensure that the patient does not ingest the medication after the use-by date.

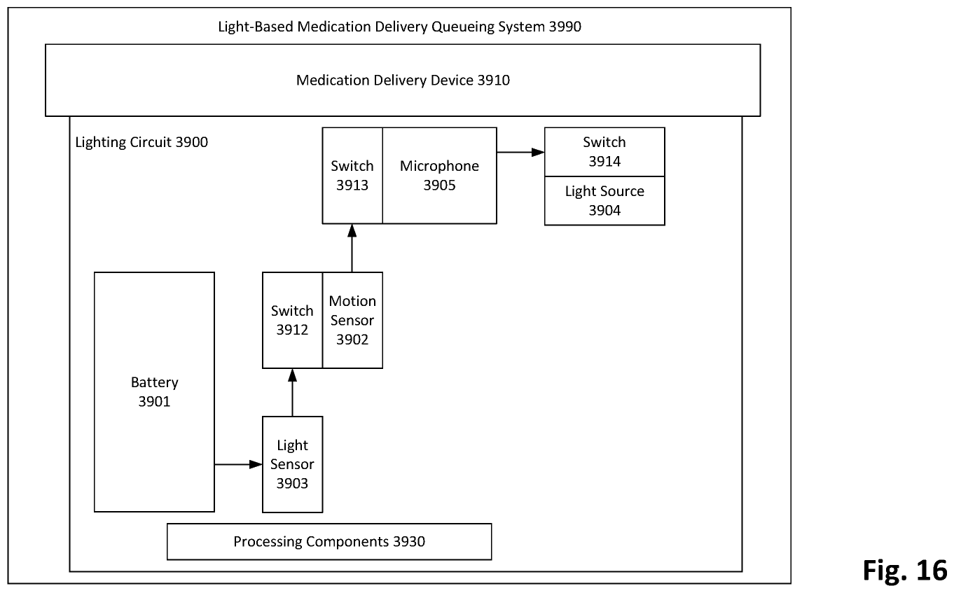

Referring now to FIG. 16, a light-based medication delivery cueing system 3990 will now be described in detail. As shown, the light-based medication delivery cueing system 3990 includes a lighting circuit 3900 and a medication delivery device 3910, such as device 300 of FIG. 10 or another medication injection device.

The lighting circuit 3900 may be integrated with the medication delivery device 3910. The term integrated with, as used herein, means that the lighting circuit 3900 is included within the medication delivery device 3910 or is otherwise directly, or indirectly (e.g., via one or more connecting or attaching components), physically connected, or attached, to (e.g., via an adhesive, etc.) the medication delivery device 3910, before and/or at the time that the medication delivery device is used by a patient (and/or by another user on the patient's behalf). Thus, battery 3901, motion sensor 3902, light sensor 3903, processing components 3930, switch 3912, switch 3913, switch 3914, microphone 3905, and light source 3904 may be integrated with the medication delivery device 3910. In some examples, the lighting circuit 3900 may be included in one or more thin and flexible adhesive labels that are attachable to, and/or attached to, the medication delivery device 3910. Thus, in some examples, any, or all, of battery 3901, motion sensor 3902, light sensor 3903, processing components 3930, switch 3912, switch 3913, switch 3914, microphone 3905, and light source 3904 may be included in one or more thin and flexible adhesive label that are attachable to, and/or attached to, the medication delivery device 3910. Additionally, in some examples, the lighting circuit 3900 may be included within the medication delivery device 3910. For example, the lighting circuit 3900 may be embedded into one or more other components of the medication delivery device 3910, such as by being molded into plastic and/or other materials of which those components may be comprised. Thus, in some examples, any, or all, of battery 3901, motion sensor 3902, light sensor 3903, processing components 3930, switch 3912, switch 3913, switch 3914, microphone 3905, and light source 3904 may be included within the medication delivery device 3910.

The lighting circuit 3900 includes a light sensor 3903, which is configured to detect the presence of light exterior to the medication delivery device 3910. The light sensor 3903 may operate in a similar manner as light sensor 3303 of FIG. 10, such as to detect light corresponding to a removal of the medication delivery device 3910 from a storage component. The lighting circuit 3900 also includes motion sensor 3902. The motion sensor 3902 may operate in a similar manner as motion sensor 3302 of FIG. 10, such as to detect motion of the medication delivery device 3910, for example as caused by a user's movement of the medication delivery device 3910.

In some examples, the motion sensor 3902 may initially be in a sleep state in which it is not powered. The motion sensor 3902 may remain unpowered until the light is detected by the light sensor 3903. When light is detected by the light sensor 3903, the motion sensor 3902 may then be powered-on to by receiving power from battery 3901. In this manner, power of the battery 3901 may be conserved. Additionally, this may prevent the motion sensor 3902 from detecting motion of the medication delivery device 3910 while the medication delivery device 3910 is still inside the storage component, which would not indicate that the user is ready to use the medication delivery device. Switch 3912 is an electrical switch that may control the flow of power from the battery 3901 to the motion sensor 3902. The switch 3912 may be initially in an open state (which does not allow power to be provided to the motion sensor 3902) and then, upon detection of the light, moved to a closed state (which does allow power to be provided to the motion sensor 3902). In some examples, the light sensor 3903 may, upon detection of the light, provide an indication (e.g., signal) of the detected light to processing components 3930, which, in turn, may provide an indication (e.g., signal) to close the switch 3912 and allow power to flow to the motion sensor 3902. In some examples, processing components 3930 (as well as other processing components described herein) may be components that are programmed, or otherwise instructed, to perform the operations that they execute as described herein, and that may be included in one or more integrated circuit (IC) chips and/or other IC components.

The lighting circuit 3900 further includes a microphone 3905 that detects at least one sound, such as may include a sound made by components of the medication delivery device 3910. In this example, power may be provided to the microphone 3905 based at least in part on a detection of the motion of the medication delivery device 3910 by motion sensor 3902. Moreover, because motion sensor 3902 is powered based on detection of the light, the microphone 3905 may also be considered to be powered based at least in part on the detection of the light and the detection of the motion. In some examples, the microphone 3905 may initially be in a sleep state in which it is not powered. The microphone 3905 may remain unpowered until the motion is detected by the motion sensor 3902. When motion is detected by the motion sensor 3902, the microphone 3905 may then be powered-on by receiving power from battery 3901. In this manner, power of the battery 3901 may be conserved. Switch 3913 is an electrical switch that may control the flow of power from the battery 3901 to the microphone 3905. The switch 3913 may be initially in an open state (which does not allow power to be provided to the microphone 3905) and then, upon detection of the motion, moved to a closed state (which does allow power to be provided to the microphone 3905). In some examples, the motion sensor 3902 may, upon detection of the motion, provide an indication (e.g., signal) of the detected motion to processing components 3930, which, in turn, may provide an indication (e.g., signal) to close the switch 3913 and allow power to flow to the microphone 3905.

In some examples, the medication delivery device 3910 may make an audible sound, such as a click sound, when injection of a medication is completed. For example, as described above, the device 300, which is an example of medication delivery device 3910, may make a click sound when injection of a medication is completed. Specifically, as described above, such as in relation to FIGS. 1A-1F, as the upper housing 308 is locked in dispensed position, such as by locking latches snapping over the latch members, an audible click may be produced that signifies to the user that the upper housing 308 has reached the dispensed position and is locked in the dispensed position

In some examples, processing components 3930 may have access to stored audio characteristics, such as a stored audio fingerprint, that correspond to an injection completion sound, such as a click sound, that is made by the medication delivery device 3910 in association with a completion of an injection. For example, these stored audio characteristics could be programmed into memory accessible to the processing components 3930, such as by a manufacturer and/or distributor of the medication delivery device 3910.

In some examples, when the microphone 3905 is powered-on, audio data corresponding to at least one sound detected by the microphone 3905 may be provided by the microphone 3905 to the processing components 3930. Upon receipt of this audio data, the processing components 3930 may determine first audio characteristics of the at least one sound detected by the microphone 3905. The processing components 3930 may then attempt to match the first audio characteristics to second audio characteristics, which are the stored audio characteristics (e.g., the stored audio fingerprint) of the the injection completion sound (e.g., click sound). When the processing components 3930 detect a sound match between the first audio characteristics of the detected sound from microphone 3905 and the second audio characteristics (e.g., the stored audio fingerprint) of the injection completion sound, then this is an indication that the injection of the medication has been completed. In some examples, the compared first and second audio characteristics may include amplitude, frequency, and the like. In some cases, the techniques used to determine the sound match may include audio classification, sound event detection, sound threshold detection, and the like. In some examples, to determine the sound match, the processing components 3930 may employ the use of frequency domain sound characteristics, time-frequency segmentation, conversion of a spectrogram into a vector and monitoring for audio characteristics that match the vector, and other techniques.

The lighting circuit 3900 further includes light source 3904, such as an OLED, OLED display, LED, etc. The light source 3904 may be activated based on a detection of a sound match by the processing components 3930. Light source 3904 may be activated by being changed to one or more active states, such as to start emitting light (e.g., by being powered-on), to start flashing, to change color (e.g., to change to a green color or another designated color associated with an active state), and the like. In the example of FIG. 16, light source 3904 may be activated by being caused to emit light, such as by being powered-on. Switch 3914 is an electrical switch that may control the flow of power from the battery 3901 to the light source 3904. The switch 3914 may be initially in an open state (which does not allow power to be provided to the light source 3904) and then, upon detection of the sound match, moved to a closed state (which does allow power to be provided to the light source 3904). In some examples, the processing components 3930 may, upon detection of the sound match, provide an indication (e.g., signal) to close the switch 3914 and allow power to flow to the light source 3904. In this manner the light source 3904 may activated based on a detection of the sound match.

In some examples, activating of light source 3904 may be a visual cue that an injection has been completed. Activation of light source 3904 may also be a visual cue to lift-up the medication delivery device 3910. In some examples, the light source 3904 may illuminate illuminable information, such as an instruction panel with words such as “injection complete” and/or “lift straight up”. It is noted that the use of the light sensor 3903 and motion sensor 3902 in lighting circuit 3900 are optional.

While example embodiments of devices for executing the disclosed techniques are described herein, the underlying concepts can be applied to any system capable of performing the techniques described herein. Thus, the methods and apparatuses described herein can be implemented, or certain aspects or portions thereof, can take the form of program code (e.g., instructions) embodied in tangible non-transitory storage media (e.g., the one or more memory components described above), including a processor-readable or machine-readable storage medium, wherein, when the program code (e.g., instructions) is loaded into and executed by a machine, the machine becomes an apparatus for performing the techniques described herein.

While the techniques described herein can be implemented and have been described in connection with the various embodiments of the various figures, it is to be understood that other similar embodiments can be used or modifications and additions can be made to the described embodiments without deviating therefrom. For example, it should be appreciated that the steps disclosed above can be performed in the order set forth above, or in any other order as desired. The techniques described herein should not be limited to any single embodiment, but rather should be construed in breadth and scope in accordance with the appended claims.

1. A light-based medication delivery cueing system comprising:
a light sensor and a motion sensor, wherein the light sensor detects light corresponding to a removal of a medication delivery device from a storage component, wherein the motion sensor detects motion of the medication delivery device; and
a first light source that is activated based at least in part on a first detection of the light and a second detection of the motion, wherein activating of the first light source is a first visual cue to perform a first medication delivery instruction.

2. The light-based medication delivery cueing system of claim 1, wherein power is provided to the motion sensor based at least in part on the first detection of the light.

3. The light-based medication delivery cueing system of claim 1, wherein the light sensor, the motion sensor and the first light source are included in one or more adhesive labels that are attached to the medication delivery device.

4. The light-based medication delivery cueing system of claim 1, wherein the light sensor, the motion sensor and the first light source are included within the medication delivery device.

5. The light-based medication delivery cueing system of claim 1, wherein the medication delivery device is a medication storage container.

6. The light-based medication delivery cueing system of claim 1, wherein the medication delivery device is a medication injection device.

7. The light-based medication delivery cueing system of claim 1, wherein the first medication delivery instruction is a cap removal instruction or a device shaking instruction.

8. The light-based medication delivery cueing system of claim 1, wherein the first light source is an organic light-emitting diode (OLED).

9. The light-based medication delivery cueing system of claim 1, wherein the first light source illuminates text.

10. The light-based medication delivery cueing system of claim 1, wherein the first light source illuminates a symbol.

11. The light-based medication delivery cueing system of claim 1, wherein the first light source is activated by at least one of causing the first light source to emit light, causing the first light source to flash, or causing the first light source to change color.

12. The light-based medication delivery cueing system of claim 1, wherein the medication delivery device further comprises a second light source, wherein activating of the second light source is a second visual cue to perform a second medication delivery instruction.

13. The light-based medication delivery cueing system of claim 12, wherein the second light source is activated based on performance of the first medication delivery instruction as detected by an additional sensor of the light-based medication cueing delivery system.

14. A light-based medication delivery cueing method comprising:
detecting, by a light sensor, light corresponding to a removal of a medication delivery device from a storage component;
detecting, by a motion sensor, motion of the medication delivery device; and
activating, based at least in part on the detecting of the light and the detecting of the motion, a first light source, wherein the activating of the first light source is a first visual cue to perform a first medication delivery instruction.

15-26. (canceled)

27. A system comprising:
a timer configured to detect an occurrence of a designated date;
a light sensor that detects light corresponding to a removal of a medication delivery device from a storage component, wherein the light sensor is powered based on a first detection of the occurrence of the designated date; and
a light source that is activated based at least in part on a second detection of the light.

28. The system of claim 27, further comprising a motion sensor that detects motion of the medication delivery device.

29. The system of claim 28, wherein the motion sensor is powered based at least in part on the second detection of the light.

30. The system of claim 28, wherein the light source is powered based at least in part on the second detection of the light and a third detection of the motion.

31-32. (canceled)

33. The system of claim 27, wherein activation of the light source causes illumination of medication delivery information.

34. The system of claim 33, wherein the medication delivery information relates to an expiration date of a medication.

35-65. (canceled)
